(19) United States
(12) Patent Application Publication
(10) Pub. No.: US 2026/0103570 A1
(43) Pub. Date: Apr. 16, 2026

(54) ANTIMICROBIAL AND/OR ANTIADHESIVE CROSSLINKING AGENTS AND ANTIMICROBIAL AND/OR ANTIADHESIVE MEDICAL HYDROGELS FORMED THEREFROM

(71) Applicant: Boston Scientific Scimed, Inc., Maple Grove, MN (US)

(72) Inventors: Philip David Dorgan, Cork (IE); Eddy Mateo Flores, Limerick (IE); Cliodhna Wallace, Cork (IE); Yen-Hao Hsu, Shrewsbury, MA (US); Heather Cirka, Hudson, MA (US); Kristen Maynard, Minneapolis, MN (US)

(73) Assignee: Boston Scientific Scimed, Inc., Maple Grove, MN (US)

(21) Appl. No.: 19/353,090

(22) Filed: Oct. 8, 2025

Related U.S. Application Data
(60) Provisional application No. 63/705,677, filed on Oct. 10, 2024.
(60) Provisional application No. 63/721,719, filed on Nov. 18, 2024.
(60) Provisional application No. 63/749,181, filed on Jan. 24, 2025.

(51) Int. Cl. C08J 3/075; A61L 27/18; A61L 27/52; A61L 27/54; C08J 3/24

(52) U.S. Cl. CPC C08J 3/075; A61L 27/18; A61L 27/52; A61L 27/54; C08J 3/246; A61L 2300/404; C08J 2379/02

(57) ABSTRACT

In some aspects, the present disclosure pertains to a system or kit for forming an antimicrobial and/or antiadhesive cross-linked hydrogel, the system or kit comprising: a multi-arm polymer comprising a core, a plurality of polymer segments having a first end that is covalently attached to the core and a second end comprising a moiety that comprises a reactive group; and an antimicrobial and/or antiadhesive multifunctional compound that comprises functional groups that are reactive with the reactive group of the multi-arm polymer.

### CROSS-REFERENCE TO RELATED APPLICATIONS

The present disclosure claims the benefit of U.S. Provisional Application No. 63/705,677 filed Oct. 10, 2024, U.S. Provisional Application No. 63/721,719 filed Nov. 18, 2024, U.S. Provisional Application No. 63/749,181 filed Jan. 24, 2025, the disclosures of which are hereby incorporated by reference.

### FIELD

The present disclosure relates to antimicrobial and/or antiadhesive crosslinking agents and antimicrobial and/or antiadhesive hydrogels formed therefrom. Hydrogels are useful, for example, in various medical applications.

### BACKGROUND

Injectable hydrogels are an emerging class of materials having a variety of medical uses. As one specific example, injectable hydrogels have been used to create or maintain space between tissues, for instance, to reduce side effects of off-target radiation therapy. For example, the hydrogel creates a space between a rectum and a prostate of a patient, moving the rectum away from the treatment region. This can help to reduce radiation exposure to the rectum and/or provide other desirable benefits.

The use of a hydrogel-based perirectal spacer material in conjunction with prostate radiation therapy is illustrated schematically in FIGS. 1A and 1B. FIG. 1A illustrates a cross-section of the human male anatomy including the prostate 110 and rectal wall 112. When the prostate is treated using radiation therapy there is a higher dose region 114 adjacent to the prostate, which is subjected to high doses of radiation, becoming a lower dose region 116 as one proceeds further from the prostate 110. As illustrated in FIG. 1B, a spacing material 118 can be injected between the prostate 110 and the rectal wall 112, which can push the rectal wall from a higher dose region 114 to a lower dose region 116, thereby reducing injury to the rectal wall.

SpaceOAR® and SpaceOAR Vue® are hydrogels that rapidly form crosslinks in-vivo. They are based on multi-arm PEG polymers having a polyol residue core functionalized with succinimidyl glutarate (SG) activated ester end groups. Above a specific pH, the SG groups will rapidly react with a trilysine acetate (a branched lysine trimer) crosslinker in-vivo to form a hydrogel. The hydrogel thus contains a residue of the multi-arm PEG having a polyol residue core (Core) functionalized with SG end groups and a residue of the trilysine acetate. For example, with reference to FIG. 2, a succinimidyl-ester-terminated multi-arm PEG polymer (210) (only one arm is illustrated) rapidly reacts with a nucleophilic amine group of crosslinking agents in the form of trilysine acetate (220) (only one amine group is illustrated) to yield a crosslinked hydrogel product (230) with crosslinks (only one crosslink is illustrated). The crosslinked hydrogel product (hydrogel) breaks down in-vivo, for instance, over the course of ca. 6-9 months before being completely expelled. The breakdown occurs primarily through the hydrolysis of the ester linkages on the glutarate groups.

However, for various reasons alternative crosslinking agents are desired for forming crosslinked hydrogels.

Furthermore, healthcare-associated infections (HAIs) are infections that patients develop while receiving healthcare treatment for other conditions. They can occur in any healthcare facility and are associated with implantable devices of all types.

There is a need in the biomedical arts for infection-resistant hydrogels, for precursors of such hydrogels, for methods of making such hydrogels and precursors, for methods of using such hydrogels and precursors, and for systems for forming such hydrogels, among other needs.

### SUMMARY

In some aspects, a kit for forming an antimicrobial cross-linked hydrogel is provided. The kit comprising: a multi-arm polymer comprising a core, a plurality of polymer segments having a first end that is covalently attached to the core and a second end comprising a moiety that comprises a reactive group; an antimicrobial multifunctional compound that comprises functional groups that are reactive with the reactive group of the multi-arm polymer.

In some aspects, which can be used in conjunction with any of the above aspects, wherein the antimicrobial multifunctional compound is a polyamine.

In some aspects, which can be used in conjunction with any of the above aspects, wherein the polyamine further comprises a cationic polymer.

In some aspects, which can be used in conjunction with any of the above aspects, wherein the cationic polymer is a natural biopolymer.

In some aspects, which can be used in conjunction with any of the above aspects, wherein the antimicrobial multifunctional compound comprises ε-poly-l-lysine n-acyl, ε-poly-l-lysine, ε-poly-l-lysine hydrochloride, ε-poly-L-lysine hydrobromide, ε-poly-L-lysine hydrofluoride, ε-poly-L-lysine hydroiodide, quaternized ε-poly-l-lysine, or any combination thereof.

In some aspects, which can be used in conjunction with any of the above aspects, wherein the kit has an absence of another multifunctional compound that comprises functional groups that are reactive with the reactive group of the multi-arm polymer.

In some aspects, which can be used in conjunction with any of the above aspects, wherein the ratio of the antimicrobial multifunctional compound is about 0.01 to about 10 moles to 1 moles of the multi-arm polymer.

In some aspects, wherein the plurality of polymer segments comprises polyethylene glycol segments.

In some aspects, which can be used in conjunction with any of the above aspects, wherein the antimicrobial multifunctional compound exhibits antibacterial properties, antifungal properties, or both.

In some aspects, which can be used in conjunction with any of the above aspects, a molar ratio of the antimicrobial compound to the multi-arm polymer is in a range from about 0.001:1 to about 10:1.

In some aspects, which can be used in conjunction with any of the above aspects, a wherein a molar ratio of the antimicrobial compound to the multi-arm polymer is in a range from about 0.2:1 to about 10:1.

In some aspects, which can be used in conjunction with any of the above aspects, wherein the antimicrobial multifunctional compound comprises about 0.01 to about 40.00 weight percent based on a total weight of the cross-linked hydrogel.

In some aspects, which can be used in conjunction with any of the above aspects, wherein the antimicrobial multifunctional compound comprises about 5 weight percent to about 40 weight percent based on a total weight of the cross-linked hydrogel.

In some aspects, which can be used in conjunction with any of the above aspects, wherein the multi-arm polymer has 3 to 16 arms.

In some aspects, which can be used in conjunction with any of the above aspects, wherein the reactive moieties of the multi-arm polymer include moieties that comprise electrophilic groups.

In some aspects, which can be used in conjunction with any of the above aspects, wherein the electrophilic groups comprise succinimide ester groups; and wherein the plurality of polymer segments comprises polyethylene glycol segments.

In some aspects, which can be used in conjunction with any of the above aspects, wherein the cross-linked hydrogel is formed in-vivo or ex-vivo and exhibits antimicrobial properties in vivo.

In some aspects a system for forming an antimicrobial cross-linked hydrogel is provided. The comprising: (a) a first composition comprising a multi-arm polymer comprising a core, a plurality of segments having a first end that is covalently attached to the core and a second end comprising a moiety that comprises a reactive group; and (b) a second composition comprising an antimicrobial multifunctional compound that comprises functional groups that are reactive with the reactive group of the multi-arm polymer, wherein the antimicrobial multifunctional compound comprises ε-poly-l-lysine n-acyl, ε-poly-l-lysine, ε-poly-l-lysine hydrochloride, ε-poly-l-lysine hydrobromide, ε-poly-l-lysine hydrofluoride, ε-poly-l-lysine hydroiodide, quaternized ε-poly-l-lysine, or any combination thereof.

In some aspects, which can be used in conjunction with any of the above aspects, wherein: the antimicrobial multifunctional compound further comprises ε-Poly-L-lysine.

In some aspects an antimicrobial cross-linked reaction product is provided. The cross-linked reaction product being formed of (a) a multi-arm polymer comprising a core, a plurality of polymer segments having a first end that is covalently attached to the core and a second end comprising a moiety that comprises a reactive group; and (b) an antimicrobial multifunctional compound that comprises functional groups that are reactive with the reactive group of the multi-arm polymer, wherein the antimicrobial multifunctional compound comprises-poly-l-lysine n-acyl, ε-poly-l-lysine, ε-poly-l-lysine hydrochloride, ε-poly-l-lysine hydrobromide, ε-poly-l-lysine hydrofluoride, ε-poly-l-lysine hydroiodide, quaternized ε-poly-l-lysine, or any combination thereof, and wherein a molar ratio of the antimicrobial compound to the multi-arm polymer is in a range from about 0.2:1 to about 10:1.

In some aspects, the present disclosure provides systems for forming hydrogels, the systems comprising (a) a quaternary-amine-containing polyamino compound that comprises a quaternary-amine-containing moiety that comprises a quaternary amine group and a polyamino moiety that comprise a plurality of primary amine groups and (b) a reactive polymer comprising a plurality of reactive moieties, wherein the reactive moieties are reactive with the plurality of primary amine groups of the quaternary-amine-containing polyamino compound, and wherein the quaternary-amine-containing moiety is coupled to the polyamino moiety through a linkage that comprises an amide group.

In some aspects, which can be used in conjunction with the above aspects, the quaternary-amine-containing moiety comprises a (triC₁₋₃₀alkyl)aminoC₂₋₃₀alkylaminocarbonyl group.

In some aspects, which can be used in conjunction with the above aspects, the quaternary-amine-containing polyamino compound is formed by a method that comprises (a) reacting a quaternary-amine-containing primary amine compound in an amide coupling reaction with an amine-protected carboxylic-acid-containing polyamino compound that comprises a carboxylic-acid group and a plurality of protected amine groups and (b) deprotecting the product of step (a) to form the quaternary-amine-containing polyamino compound. In some of these aspects, the quaternary-amine-containing primary amine compound is a C₂₋₃₀alkanediamine compound that comprises first and second amine groups, wherein the first amine group is a primary amine group and the second amine group is a quaternary amine group that contains three C₁₋₃₀alkyl groups, which can be the same or different.

In some aspects, which can be used in conjunction with the above aspects, the quaternary-amine-containing primary amine compound is formed by a method that comprises (a) reacting a C₁₋₃₀haloalkane in a substitution reaction with a protected C₂₋₃₀alkanediamine compound that comprises first and second amine groups in which the first amine group is a protected primary amine group and the second amine group is a tertiary amine group that contains two C₁₋₃₀alkyl groups, which can be the same or different, and (b) deprotecting the product of step (a) to form the quaternary-amine-containing primary amine compound.

In some aspects, which can be used in conjunction with the above aspects, the amine-protected carboxylic-acid-containing polyamino compound is a poly(amino acid) that comprises a plurality of protected primary-amine-containing side groups.

In some aspects, which can be used in conjunction with the above aspects, the quaternary-amine-containing moiety is a zwitterionic quaternary-amine-containing moiety that comprises the quaternary amine group and a sulfonate group. In some of these aspects, the zwitterionic quaternary-amine-containing moiety comprises a (diC₁₋₃₀alkyl)(sulfoC₁₋₃₀alkyl)aminoC₂₋₃₀alkylaminocarbonyl group.

In some aspects, which can be used in conjunction with the above aspects, the quaternary-amine-containing polyamino compound is formed by a method that comprises (a) reacting a zwitterionic quaternary-amine-containing primary amine compound in an amide coupling reaction with an amine-protected carboxylic-acid-containing polyamino compound that comprises a carboxylic-acid group and a plurality of protected amine groups and (b) deprotecting the product of step (a) to form the quaternary-amine-containing polyamino compound. In some of these aspects, the zwitterionic quaternary-amine-containing primary amine compound is a zwitterionic C₂₋₃₀alkanediamine compound that comprises first and second amine groups in which the first amine group is a primary amine group and second amine group is a quaternary amine group that contains two C₁₋₃₀alkyl groups, which can be the same or different, and one sulfoC₁₋₃₀alkyl group.

In some aspects, which can be used in conjunction with the above aspects, the zwitterionic quaternary-amine-containing primary amine compound is formed by a method that comprises (a) reacting a cyclic ester of a C₁₋₃₀ hydroxyl sulfonic acid with a protected C₂₋₃₀alkanediamine compound that comprises first and second amine groups in which the first amine group is a protected primary amine group and the second amine group is a tertiary amine group that contains two C₁₋₃₀alkyl groups, which can be the same or different and (b) deprotecting the product of step (a) to form the zwitterionic quaternary-amine-containing primary amine compound.

In some aspects, which can be used in conjunction with the above aspects, the amine-protected carboxylic-acid-containing polyamino compound is a poly(amino acid) that comprises a plurality of protected primary-amine-containing side groups.

In some aspects, which can be used in conjunction with the above aspects, the quaternary amine group comprises an iodide counter-anion.

In some aspects, which can be used in conjunction with the above aspects, the plurality of primary amine groups comprises —(CH₂)ₓ—NH₂ groups where x is 1, 2 3, 4, 5 or 6.

In some aspects, which can be used in conjunction with the above aspects, at least a portion of the plurality of primary amine side groups are lysine residues.

In some aspects, which can be used in conjunction with the above aspects, the reactive polymer is a reactive multi-arm polymer that comprises a plurality of hydrophilic polymer arms comprising hydrophilic polymer segments and reactive end groups that covalently crosslink with at least a portion of the plurality of primary amine groups of the quaternary-amine-containing polyamino compound.

In some aspects, which can be used in conjunction with the above aspects, the hydrophilic polymer segments are selected from polyalkylene oxide segments, polyester segments, polyoxazoline segments, polydioxanone segments, and polypeptide segments.

In some aspects, which can be used in conjunction with the above aspects, the reactive groups are electrophilic groups selected from imidazole esters, imidazole carboxylates, benzotriazole esters, or imide esters. In some of these aspects, the electrophilic groups are cyclic imide ester groups.

In other aspects, the present disclosure pertains to medical hydrogels formed by crosslinking quaternary-amine-containing polyamino compound in accordance with any of the above aspects with a reactive polymer in accordance with any of the above aspects.

In further aspects, the present disclosure pertains to methods of treatment comprising administering to a subject a mixture that comprises a quaternary-amine-containing polyamino compound in accordance with any of the above aspects and a reactive polymer in accordance with any of the above aspects under conditions such that the quaternary-amine-containing polyamino compound and the reactive polymer form covalent crosslinks after administration.

As seen from the foregoing, in various aspects, the present disclosure pertains to quaternary-amine-containing polyamino compounds, which can act as crosslinking agents for the formation of hydrogels, and which are anticipated to provide various hydrogels with antimicrobial and/or anti-adhesive properties. In this regard, compounds that contain quaternary amines (also referred to as quaternary ammonium compounds) have been reported to have antimicrobial properties, while being non-cytotoxic to human cells, and zwitterionic compounds that contain quaternary amines have been reported to have anti-adhesive/anti-fouling properties. See, e.g., Zander Z K and Becker M L. Antimicrobial and Antifouling Strategies for Polymeric Medical Devices. ACS Macro Lett. 2018 Jan. 16; 7(1): 16-25; Zander Z K, et al. Post-fabrication QAC-functionalized thermoplastic polyurethane for contact-killing catheter applications. Biomaterials. 2018 September; 178:339-350; Dreger N Z, et al. Zwitterionic amino acid-based Poly(ester urea)s suppress adhesion formation in a rat intra-abdominal cecal abrasion model. Biomaterials. 2019 November; 221:119399.

The above and other aspects, embodiments, features and benefits of the present disclosure will be readily apparent from the following detailed description.

### BRIEF DESCRIPTION OF THE DRAWINGS

FIG. 1A illustrates a cross-section of the human male anatomy including the prostate and rectal wall.

FIG. 1B illustrates an example of a spacing material between the prostate and the rectal wall.

FIG. 2 schematically illustrates an example process for forming a prior art a crosslinked hydrogel product.

FIG. 3 schematically illustrates an example process for forming a cross-linked hydrogel, in accordance with an embodiment of the present disclosure.

FIG. 4 illustrates a delivery device, in accordance with an embodiment of the present disclosure.

FIG. 5 illustrates a delivery device, in accordance with another embodiment of the present disclosure.

FIG. 6 schematically illustrates a method of forming a quaternary-amine-containing polyamino compound, in accordance with an embodiment of the present disclosure.

FIG. 7 schematically illustrates a method of forming a zwitterionic quaternary-amine-containing polyamino compound, in accordance with an embodiment of the present disclosure.

FIG. 8 schematically illustrates a crosslinking reaction between a reactive multi-arm polymer and a quaternary-amine-containing polyamino compound, in accordance with an embodiment of the present disclosure.

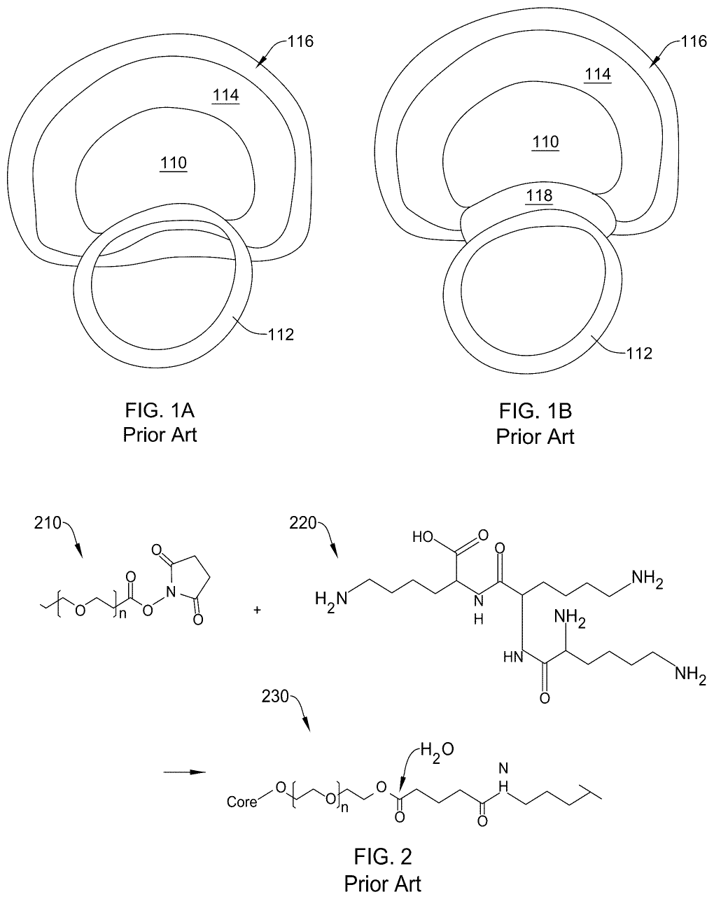

### DETAILED DESCRIPTION

In some aspects of the present disclosure provides kits and systems for forming antimicrobial cross-linked medical hydrogels. The kits and systems herein can comprise (a) a multi-arm polymer comprising a core, a plurality of polymer segments having a first end that is covalently attached to the core and a second end comprising a moiety that comprises a reactive group; and (b) an antimicrobial multifunctional compound that comprises functional groups that are reactive with the reactive group of the multi-arm polymer, as described herein.

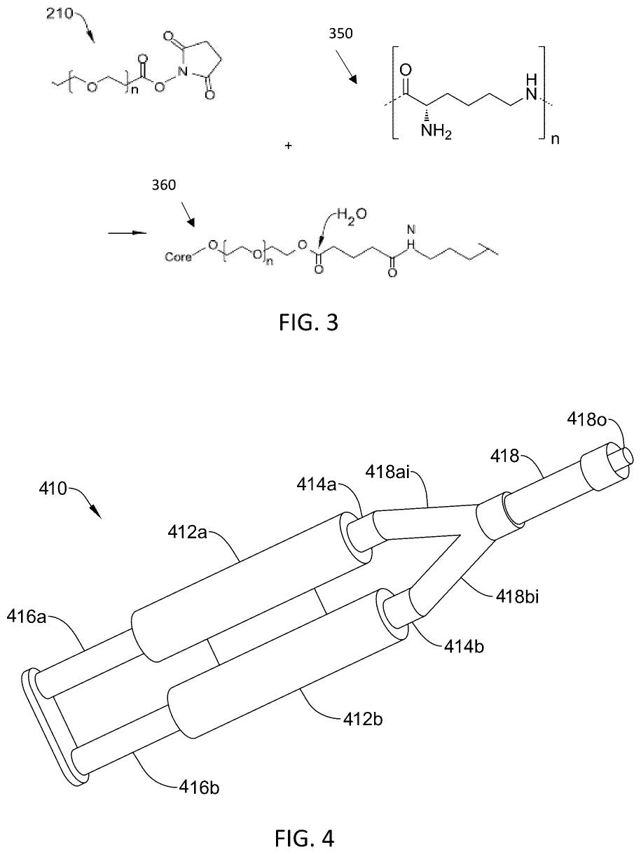

For instance, the antimicrobial multifunctional compound can comprise a natural biopolymer such as ε-poly-l-lysine n-acyl, ε-poly-l-lysine, ε-poly-l-lysine hydrochloride, ε-poly-l-lysine hydrobromide, ε-poly-l-lysine hydrofluoride, ε-poly-l-lysine hydroiodide, quaternized ε-poly-l-lysine, or any combination thereof, as detailed herein. For example, FIG. 3 schematically illustrates an example process for forming a cross-linked hydrogel 360 in accordance with an embodiment of the present disclosure. As illustrated in FIG. 3, a multi-arm polymer 210 (e.g., the 8-arm polymer formed with PEG linkages and succinimidyl glutarate end groups) can be reacted with an antimicrobial multifunctional compound 350 (e.g., ε-poly-l-lysine) to yield the cross-linked hydrogel 360. That is, the approaches herein provide alternative crosslinkers for forming cross-linked medical hydrogels having similar cross-linking properties as compared to hydrogels formed from traditional crosslinkers (e.g., traditional multifunctional compounds such as TLA), and yet also exhibit antimicrobial properties.

Moreover, it may be desired to incorporate an antimicrobial agent (e.g., antimicrobial polymers) in a hydrogel composition, for instance, to add an inherent antimicrobial effect and achieve localized antimicrobial effect through surface-based interactions with microorganisms, significantly reducing most side effects of a systemic application, including lower generation of resistance. Generally, antimicrobial agents exhibit an ability to kill or inhibit the growth of microorganisms such as bacteria, viruses, fungi, and protozoa. Materials or compounds with antimicrobial properties can either destroy these microorganisms (bactericidal, viricidal, fungicidal) or suppress their ability to grow and reproduce (bacteriostatic, fungistatic). For instance, the antimicrobial properties of EPL (and derivatives thereof) may be manifested by altering the integrity and permeability of the microorganisms' cell membrane through electrostatic interactions of the positively charged side chains of EPL and the negatively charged membrane. EPL in solution concentrates around the bacterial cell wall through electrostatic-driven diffusion and permeates the bacterial cell wall inducing pore formation through a carpet-like mechanism. The intracellular content from the microorganisms is leaked through the formed pores, the instability of the membrane causes the cell architecture to collapse ultimately generating cell death. In some embodiments, the antimicrobial multifunctional compound can itself provide the desired antimicrobial properties (in addition to providing a crosslinking function) and thereby can negate any need for additional antimicrobial agents and/or crosslinkers.

Additionally, EPL is a natural biopolymer that is readily soluble in water, but insoluble in non-polar organic solvents. EPL and its derivatives show great heat stability and can undergo autoclave conditions without degradation, which can be beneficial in the context of medical applications such as medical hydrogels. EPL may be susceptible to enzyme degradation, for instance, from Streptomyces peptidase. The amine side chain of EPL provides a reactive site for ionic or covalent bonding, thereby facilitating crosslinking with other polymers, as well as to generate EPL-based derivatives through different chemistries to modulate the bioactivity of the material as may be desired in some instances.

Accordingly, the present disclosure is directed to forming cross-linked hydrogels with EPL and/or derivatives thereof. The EPL and/or derivatives can be employed as an additive (in addition to traditional amounts of a TLA crosslinker) or can be employed as a substitute or replacement for TLA (e.g., in an absence of the TLA crosslinker), as described herein. For instance, EPL and/or derivatives thereof can be present in higher amounts (e.g., greater than about 1 percent, about 2 percent or about 5 percent based on a total weight of a cross-linked hydrogel) than would typically be employed merely in an additive context (e.g., as an antimicrobial additive), such that the EPL and/or derivatives thereof can provide the dual functions of an antimicrobial agent and a primary or sole cross-linking agent (e.g., as a primary or sole crosslinker) for formation of the antimicrobial cross-linked hydrogels, described herein. Notably, the resultant cross-linked hydrogels herein (e.g., which are crosslinked or formed in vivo) can exhibit antimicrobial properties (e.g., in-vivo), and yet can also exhibit other properties (e.g., rigidity (e.g., in terms of Young's modulus), gelation times) that are similar to or improved relative to traditional crossed-linked hydrogels formed with TLA (e.g., formed with solely with a TLA cross-linker in an absence of EPL and/or derivatives thereof), as described herein. For instance, despite utilizing the same cross-linking sites (e.g., SG sites), the resultant cross-linked polymers herein that are formed with the EPL can have similar or improved properties (e.g., improved rigidity as measured in Young's modulus) relative to traditional cross-linked hydrogels formed with TLA (e.g., formed entirely solely with a TLA crosslinker) e.g., due to structural differences between the EPL and TLA molecules.

In the present disclosure, crosslinked hydrogels products are formed from a multi-arm polymer comprising a core, a plurality of segments having a first end that is covalently attached to the core and a second end comprising a moiety that comprises a reactive group. Examples of suitable multi-arm polymers (e.g., PEG polymers), are described herein. The multi-arm polymer is reacted with at least an antimicrobial multifunctional compound that comprises functional groups that are reactive with the reactive group of the multi-arm polymer (e.g., PEG).

In various embodiments, the antimicrobial multifunctional compound (e.g., for use in the second compositions of the above systems and devices), and for use in forming the above crosslinked products, may be a polyamine. In some embodiments, the polyamine further comprises a cationic polymer. In some embodiments, the cationic polymer is a natural biopolymer. For instance, in some examples the antimicrobial multifunctional compound comprises a natural biopolymer including ε-poly-l-lysine

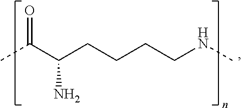

wherein n is a value in a range from 10 to 40 or is a value in a range from 27 to 35, or derivatives thereof. For example, in some embodiments the antimicrobial multifunctional compound comprises: ε-poly-l-lysine n-acyl, ε-poly-l-lysine, ε-poly-l-lysine hydrochloride, ε-poly-l-lysine hydrobromide, ε-poly-l-lysine hydrofluoride, ε-poly-l-lysine hydroiodide, quaternized ε-poly-l-lysine, or any combination thereof.

In some embodiments, the kits and crosslinked products formed therefrom include an absence of another multifunctional compounds (e.g., an absence of any traditional multifunctional compounds). Particular examples of multifunctional amines which are traditional multifunctional compounds include trilysine, ethylenetriamine, diethylene triamine, hexamethylenetriiamine, di(heptamethylene) triamine, di(trimethylene) triamine, bis(hexamethylene) triamine, triethylene tetramine, tripropylene tetramine, tetraethylene pentamine, hexamethylene heptamine, pentaethylene hexamine, dimethyl octylamine, dimethyl decylamine, and JEFFAMINE polyetheramines available from Huntsman Corporation, among others.

In some embodiments, the molar ratio of the antimicrobial compound to the multi-arm polymer is in a range from about 0.001:1 to about 10:1, from about 0.001:1 to about 0.2:1, or from 0.2:1 to about 10:1. For instance, having the antimicrobial compound present in a molar ratio in a range from 0.2:1 to about 10:1 ensures that the antimicrobial compound present at bio effective levels (e.g., to exhibit antimicrobial properties in-vivo such as antibacterial properties, antifungal properties, or both) and yet also exhibits a crosslinking function for forming a hydrogel, as described herein.

In some embodiments, the antimicrobial multifunctional compound can comprise about 0.01 weight percent (e.g., corresponding to a molar ratio of the antimicrobial compound to the multi-arm polymer of 0.001:1) to about 40.00 weight percent (e.g., corresponding to a molar ratio of the antimicrobial compound to the multi-arm polymer of about 0.2:1) based on a total weight of the cross-linked hydrogel. For instance, the antimicrobial multifunctional compound can be ε-Poly-L-lysine and/or a derivative thereof that comprises 0.01 to about 40.00 weight percent based on a total weight of the cross-linked hydrogel. In some embodiments, the antimicrobial multifunctional compound can comprise about 1 to about 40 weight percent, about 2 to about 40 weight percent, about 3 to about 40 weight percent, about 4 to about 40 weight percent, about 5 to about 40 weight percent, about 10 to about 40 weight percent, about 20 to about 40 weight percent, about 5 to about 30 weight percent, or about 10 to about 20 weight percent based on a total weight of the cross-linked hydrogel. In some instances, the antimicrobial multifunctional compound can be ε-Poly-L-lysine and/or a derivative thereof that comprises from about 0.1 to about 40 weight percent based on a total weight of the cross-linked hydrogel, for example, ranging from 0.1 to 0.25 to 0.5 to 1.0 to 2.5 to 5.0 to 10 to 25 to 40 weight percent based on a total weight of the cross-linked hydrogel (in other words, ranging between any two of the preceding values). In some instances, the antimicrobial multifunctional compound can be ε-Poly-L-lysine and/or a derivative thereof that comprises from about 0.1 to about 2 weight percent based on a total weight of the cross-linked hydrogel, for example, ranging from 0.1 to 0.2 to 0.3 to 0.4 to 0.5 to 0.6 to 0.7 to 0.8 to 0.9 to 1.0 to 1.2 to 1.4 to 1.6 to 1.8 to 2.0 weight percent based on a total weight of the cross-linked hydrogel. In some instances, the antimicrobial multifunctional compound can be ε-Poly-L-lysine and/or a derivative thereof that comprises from about 10 to about 30 weight percent based on a total weight of the cross-linked hydrogel, for example, ranging from 10 to 12 to 14 to 16 to 18 to 20 to 22 to 24 to 26 to 28 to 30 weight percent based on a total weight of the cross-linked hydrogel.

The antimicrobial multifunctional compound can be employed as a replacement for TLA (e.g., with an absence of the TLA crosslinker). In some embodiments, the kits, systems, and resultant crosslinked hydrogels formed therefrom can include an absence of another cross-linker (e.g., an absence of TLA) other than EPL or a derivative thereof. In such instances, the antimicrobial multifunctional compound can be present as the sole (only) cross-linker that reacts with the multi-arm polymer. Stated differently, the antimicrobial multifunctional compound can act as a replacement for traditional multifunctional compound (e.g., TLA) typically employed as a cross-linker for forming the cross-linked hydrogel product. In such embodiments, the antimicrobial multifunctional compound can alone comprise about 30 weight percent, about 35 weight percent, or about 40 weight percent of the based on a total weight of the cross-linked hydrogel. In such embodiments, antimicrobial multifunctional compound and the multi-arm polymer can comprise 100 weight percent, about 99 weight percent, about 98 weight percent, about 97 weight percent, about 96 weight percent, about 95 weight percent or about 90 weight percent based on a total weight of the cross-linked hydrogel. The remainder of the weight of the cross-linked hydrogel can be manifested as one or more agents (e.g., therapeutic agents and imaging agents) such as those described herein.

Reactive polymers for use in the present disclosure include reactive multi-arm polymers that comprise a plurality of polymer arms linked to a core region, at least a portion of the arms comprising a hydrophilic polymer segment. One end of the hydrophilic polymer segment is covalently linked to the core region and an opposite end of the hydrophilic polymer segment is covalently linked to a reactive moiety.

In certain embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a reactive moiety that is covalently linked to the second end of the cyclic anhydride residue.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, having a number of arms ranging between any two of the preceding values).

Reactive moieties include moieties that comprise electrophilic groups. Electrophilic groups may be selected, for example, from cyclic imide ester groups, such as succinimide ester groups,

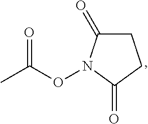

maleimide ester groups, glutarimide ester groups, diglycolimide ester groups, phthalimide ester groups, and bicyclo[2.2.1]hept-5-ene-2,3-dicarboxylic acid imide ester groups,

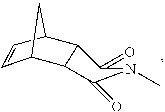

imidazole ester groups, imidazole carboxylate groups and benzotriazole ester groups, among other possibilities.

The electrophilic groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 10 and 1000 monomer units or more.

In certain embodiments, the core region comprises a residue of a polyol comprising three or more hydroxyl groups, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl) ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In some embodiments, the multi-arm polymers of the present disclosure may be radiopaque. One way to introduce radiopacity to the multi-arm polymers is to employ an iodine-containing polyol when forming the multi-arm polymers. Illustrative iodinated polyols include iodine-containing polyols that are known for use as iodinated contrast agents, such as 1,3,5-triiodo-2,4,6-tris-hydroxymethylbenzene, iodixanol, iotrolan, iohexol, ioversol, iopamidol, iohexol impurity J, and iopromide, among others.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The T₈ cage-like silicon-oxygen cores are widely studied and have the formula [RSiO_{3/2}]₈, or equivalently R₈Si₈O₁₂. Such a structure is shown here:

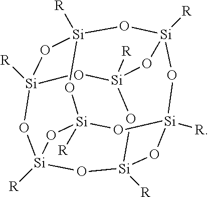

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some embodiments of the present disclosure, a polyol such as one of those described below, among others, may be used as multi-functional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, USA and Sigma Aldrich.

In some of these embodiments, the hydroxy-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

In some embodiments, the multi-arm polymers of the present disclosure may be radiopaque. One way to introduce radiopacity to the multi-arm polymers is to employ an iodine-containing cyclic anhydride when forming the multi-arm polymers. Illustrative iodine-containing cyclic anhydrides for use in the present disclosure include 4-(2,3,5-triiodophenyl)tetrahydropyran-2,6-dione, 4-(2-iodophenyl)tetrahydropyran-2,6-dione, 4-(3-iodophenyl)tetrahydropyran-2,6-dione, 4-(4-iodophenyl)tetrahydropyran-2,6-dione, 4-((4-iodophenyl)methyl), tetrahydropyran-2,6-dione, 4-1(2-iodophenyl)methyl, tetrahydropyran-2,6-dione, 3-(2-iodophenyl)tetrahydrofuran-2,5-dione, 3-(3-iodophenyl)tetrahydrofuran-2,5-dione, 3-(4-iodophenyl)tetrahydrofuran-2,5-dione, 3-1(2-iodophenyl)methyltetrahydrofuran-2,5-dione, and 3-((4-iodophenyl)methyl)tetrahydrofuran-2,5-dione, among others.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group. Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA and Sigma Aldrich, and can be used to form hydrolysis-resistant multi-arm polymers.

A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In some aspects of the present disclosure, a system is provided that comprises (a) a multi-arm polymer (e.g., a reactive multi-arm polymer) comprising a core, a plurality of segments having a first end that is covalently attached to the core and a second end comprising a moiety that comprises a reactive group, as described herein and (b) a second composition that comprises an antimicrobial multifunctional compound that comprises functional groups that are reactive with the reactive group of the multi-arm polymer, as described herein, wherein the system is configured to deliver the multi-arm polymer and the antimicrobial multifunctional compound under conditions such that covalent crosslinks are formed between the reactive multi-arm polymer and the antimicrobial multifunctional compound.

The first composition may be a first fluid composition comprising the multi-arm polymer a first dry composition that comprises the multi-arm polymer, to which a suitable fluid such as water for injection, saline, etc. can be added to form a first fluid composition. In addition to the multi-arm polymer, the first composition may further comprise additional agents, including therapeutic agents, imaging agents, colorants, tonicity adjusting agents, suspension agents, wetting agents, and pH adjusting agents as described below.

The second composition may be a second fluid composition comprising the antimicrobial multifunctional compound, or a second dry composition that comprises the antimicrobial multifunctional compound, to which a suitable fluid such as water for injection, saline, etc. can be added to form a second fluid composition. In addition to the antimicrobial multifunctional compound, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the multi-arm polymer with a second fluid comprising the antimicrobial multifunctional compound. Upon mixing the first and second fluid compositions, the antimicrobial multifunctional compound crosslinks with the reactive multi-arm polymer, forming a crosslinked product. The first and second fluid compositions may be combined form crosslinked hydrogels, either in vivo or ex vivo.

In some embodiments, the antimicrobial multifunctional compound is initially combined with the reactive multi-arm polymer under conditions where crosslinking between the reactive multi-arm polymer and the antimicrobial multifunctional compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the antimicrobial multifunctional compound and the reactive multi-arm polymer, thereby forming a crosslinked product.

In some embodiments, the system comprises (a) a first composition that comprises the reactive multi-arm polymer, as described hereinabove, (b) a second composition that comprises first composition that antimicrobial multifunctional compound, as described hereinabove, and, optionally, (c) a third composition, specifically, an accelerant composition, that contains an accelerant that is configured to accelerate a crosslinking reaction between the first composition and the second composition.

The second composition may be a second fluid composition comprising the antimicrobial multifunctional compound that is buffered to an acidic pH or a second dry composition that comprises the antimicrobial multifunctional compound, to which a suitable fluid such as water for injection, saline, an acidic buffer solution, etc. can be added to form a second fluid composition comprising the antimicrobial multifunctional compound that is buffered to an acidic pH. In some embodiments, for example, the acidic buffering composition may comprise monobasic sodium phosphate, among other possibilities. The second fluid composition comprising the comprising the antimicrobial multifunctional compound may have a pH ranging, for example, from about 3 to about 5. In addition to the comprising the antimicrobial multifunctional compound, the second composition may further comprise additional agents, including therapeutic agents, imaging agents, colorants, tonicity adjusting agents, suspension agents, wetting agents, and pH adjusting agents as described below.

The first composition may be a first fluid composition comprising the reactive polymer or a first dry composition that comprises the reactive polymer from which a fluid composition is formed, for example, by the addition of a suitable fluid such as water for injection, saline, or the second fluid composition comprising the antimicrobial multifunctional compound that is buffered to an acidic pH. In addition to the reactive polymer, the first composition may further comprise additional agents, including therapeutic agents, imaging agents, colorants, tonicity adjusting agents, suspension agents, wetting agents, and pH adjusting agents as described below.

In a particular embodiment, the second composition is a second fluid composition comprising the antimicrobial multifunctional compound that is buffered to an acidic pH and the first composition comprises a dry composition that comprises the reactive polymer. The second composition may then be mixed with the first composition to provide a prepared fluid composition that is buffered to an acidic pH and comprises the antimicrobial multifunctional compound and the reactive polymer. In a particular example, a syringe may be provided that contains the second fluid composition comprising the antimicrobial multifunctional compound that is buffered to an acidic pH, and a vial may be provided that comprises the dry composition (e.g., a powder) that comprises the reactive polymer. The syringe may then be used to inject the second fluid composition into the vial containing the reactive polymer to form a prepared fluid composition that is buffered to an acidic pH and contains the antimicrobial multifunctional compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the antimicrobial multifunctional compound and the reactive polymer as described above, and a fluid accelerant composition that is buffered to basic pH as described above, may be combined form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, additional antimicrobials (e.g., in addition to EPL and/or derivatives thereof), analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering the first and second compositions, as described herein, within a subject.

In some embodiments, the system may include a delivery device that comprises a first reservoir that contains a first fluid composition that comprises a reactive multi-arm polymer as described herein and a second reservoir that contains a second fluid composition that comprises an antimicrobial multifunctional compound, as described herein, wherein the first and second fluid compositions form a crosslinked product upon mixing.

In particular embodiments, and with reference to FIG. 4, the system may include a delivery device 410 that comprises a double-barrel syringe, which includes a first barrel 412a having a first barrel outlet 414a, which first barrel contains the first composition, a first plunger 416a that is movable in the first barrel 412a, a second barrel 412b having a second barrel outlet 414b, which second barrel 412b contains the second composition, and a second plunger 416b that is movable in the second barrel 412b. In some embodiments, the device 410 may further comprise a mixing section 418 having a first mixing section inlet 418ai in fluid communication with the first barrel outlet 414a, a second mixing section inlet 418bi in fluid communication with the second barrel outlet, and a mixing section outlet 4180.

In some embodiments, the delivery device may further comprise a needle, cannula or catheter tube that is configured to receive first and second fluid compositions from the first and second barrels. For example, a needle, cannula or catheter tube may be configured to form a fluid connection with an outlet of a mixing section by attaching the needle, cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof be injected as a scaffold, and/or the first and second fluid compositions or a fluid admixture thereof can be injected as a carrier of therapeutic agents in the treatment of diseases and cancers and the repair and regeneration of tissue, among other uses.

After administration of the compositions of the present disclosure (either separately as first and second fluid compositions that mix in vivo or as a fluid admixture of the first and second fluid compositions) a crosslinked hydrogel is ultimately formed at the administration location.

In some embodiments, during or after administration the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may vary widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel composition to a subject. Such systems may include one or more of the following: a syringe barrel, which may or may not contain a crosslinked hydrogel composition as described herein; a vial, which may or may not contain a crosslinked hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel composition may be provided in dry form (e.g., powder form) or in a form that is ready for injection, such as an injectable hydrogel form (e.g., a suspension of crosslinked hydrogel particles).

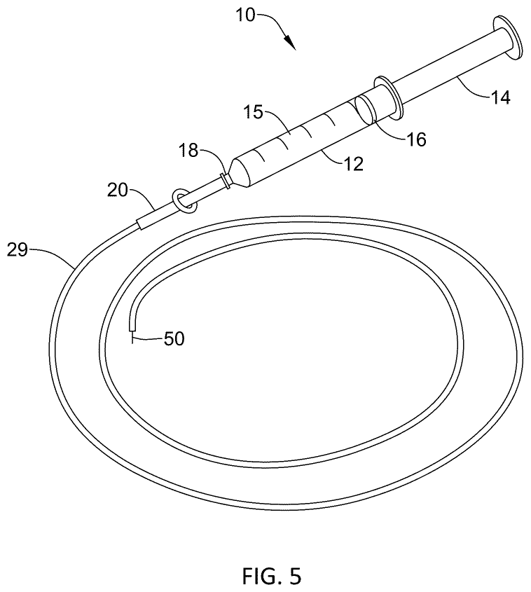

FIG. 5 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes including those described herein. For instance, crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

In further aspects, the present disclosure pertains to quaternary-amine-containing polyamino compounds (i.e., compounds that comprise one or more quaternary amine groups and a plurality of amino (—NH₂) groups) that comprise a polyamino moiety that is coupled to a quaternary-amine-containing moiety. In some embodiments, the quaternary-amine-containing polyamino compounds comprise a polyamino moiety that is coupled to a quaternary-amine-containing moiety through an amide-containing linkage. Amide-containing linkages are advantageous in cases where water stability of the coupling between the polyamino moiety and the quaternary-amine-containing moiety, relative to other couplings such as ester couplings, is desired. Such quaternary-amine-containing polyamino compounds are envisioned to confer antimicrobial and/or anti-adhesive properties upon crosslinked products that are formed using the same.

Polyamino moieties in accordance with the present disclosure include polyamino moieties having a plurality (two, three, four, five, six, seven, eight, nine, ten or more) primary amino groups. For example, the polyamino moieties may comprises a plurality (two, three, four, five, six, seven, eight, nine, ten or more) of —(CH₂)ₓ—NH₂ groups where x is 0, 1, 23, 4, 5 or 6. In some of these embodiments, the polyamino moieties may comprises a plurality of —(CH₂)ₓ—NH₂ groups disposed along a polymeric moiety (defined herein as a moiety comprising 2, 3, 4, 5, 6, 7, 8, 9, 10 or more monomer residues), which may be for example, a polyamide moiety (e.g., a polypeptide formed from amino acid resides), a polyalkylene moiety, or a polysaccharide moiety, among others.

Quaternary-amine-containing moieties in accordance with the present disclosure include quaternary-amine-containing moieties that comprise at least one quaternary amine group.

Quaternary-amine-containing moieties in accordance with the present disclosure include zwitterionic quaternary-amine-containing moieties that comprise at least one quaternary amine group and at least one sulfonate group.

In some embodiments, quaternary-amine-containing polyamino compounds in accordance with the present disclosure are prepared by a synthesis procedure in which a quaternary-amine-containing primary amine compound comprising a primary amine group and at least one quaternary amine group is reacted with an amine-protected carboxylic-acid-containing polyamino compound comprising a carboxylic-acid group and a plurality of protected amine groups. In some embodiments, the quaternary-amine-containing primary amine compound is a zwitterionic quaternary-amine-containing primary amine compound comprising a primary amine group, at least one quaternary amine group, and at least one sulfonate group.

Carboxylic-acid-containing polyamino compounds for use in the present disclosure include poly(amino acids) that comprise a plurality of primary-amine-containing side groups (e.g., two, three, four, five, six, seven, eight, nine, ten, or more primary-amine-containing side groups), such as, for example, poly(amino acids) that comprise lysine and/or ornithine. Examples include poly(amino acids) that contain two, three, four, five, six, seven, eight, nine, ten, or more lysine and/or ornithine amino acid residues, including polylysines (e.g., dilysine, trilysine, tetralysine, pentalysine, hexalysine, etc.), polyornithines (e.g., diornithine, triornithine, tetraornithine, pentaornithine, hexaornithine, etc.), and poly(lysine-co-ornithines). Carboxylic-acid-containing polyamino compounds for use in the present disclosure further include carboxylic-acid-terminated polyamines such as carboxylic-acid-terminated poly(allyl amines), carboxylic-acid-terminated poly(vinyl amines), or carboxylic-acid-terminated chitosan.

Some commercially available examples of carboxylic-acid-substituted polyamino compounds also include 16-amino-3-[2-[(4-aminobutyl)(3-aminopropyl)amino]-2-oxoethyl]-12-(3-aminopropyl)-6,9-bis(carboxymethyl)-11-oxo-3,6,9,12-tetraazahexadecanoic acid, L-ornithyl-L-ornithyl-L-ornithine, N²-[1-[N²—[N²—(N-L-valyl-L-alanyl)-L-lysyl]-L-lysyl]-L-prolyl]-L-Lysine, L-Lysyl-L-tryptophyl-L-lysyl-L-lysine, N²,N⁵,N⁵-tris(3-aminopropyl)-L-ornithine, L-lysyl-L-ornithyl-L-lysine, D-lysyl-D-lysyl-D-lysine, L-lysyl-L-lysyl-L-lysine, glycylglycyl-L-lysylglycylglycyl-L-lysine, N²—[N⁴—[N—[N—(N-glycylglycyl)glycyl]glycyl]-L-lysyl]-L-lysine, L-Lysyl-L-threonyl-L-lysyl-L-lysine, glycylglycyl-L-lysyl-L-lysylglycyl-L-cysteine, L-lysyl-L-arginyl-L-lysyl-L-lysine, L-arginyl-L-lysyl-L-lysyl-L-lysine, L-leucyl-L-lysyl-L-seryl-L-lysyl-L-lysine, L-alanyl-L-methionylglycyl-L-lysyl-L-lysyl-L-lysine, L-lysyl-L-lysyl-L-lysyl-L-arginyl-L-glutamine, L-seryl-L-isoleucyl-L-lysyl-L-lysyl-Llysyl-L-lysine, N²—(N²-L-ornithyl-L-lysyl)-L-lysine, lysyllysyl-lysine, and L-lysyl-L-lysyl-L-lysyl-L-alanine.

Examples of protective groups for use in protecting the amine groups of the carboxylic-acid-containing polyamino compound include tert-butoxycarbonyl (Boc) groups, 9-fluorenylmethoxycarbonyl (Fmoc) groups, benzyloxycarbonyl (Cbz) groups, acetamide (Ac) groups, triphenylmethylamine (Tr) groups, trifluoroacetyl (TFA) groups, 6-nitroveratryloxycarbonyl (Nvoc) groups, among others.

In some embodiments, the quaternary-amine-containing polyamino compounds may be radiopaque. One way to introduce radiopacity is to include one or more radiopaque amino acids in the carboxylic-acid-containing polyamino compound. For example, where radiopacity is desired, iodinated amino acids may be included in the above-described poly(amino acids). Specific examples of iodinated amino acids include iodo-phenylalanine, diiodo-phenylalanine, iodotyrosine, diiodotyrosine, iodothyronine, diiodothyronine, triiodothyronine also known as T3, tetraiodothyronine also known as thyroxine or T4, and iodo-L-DOPA, among others.

Quaternary-amine-containing primary amine compounds comprising a primary amine group and at least one quaternary amine group for use in the present disclosure include quaternary-amine-containing primary amine compounds that comprise a C₂₋₃₀alkanediamine compound in which one amine group is a primary amine group and the other amine group is substituted with three C₁₋₃₀alkyl groups, which can be the same or different.

Examples of C₂₋₃₀alkanediamine compounds include ethanediamines, propanediamines, butanediamines, pentanediamines, hexanediamines, heptanediamines, octanediamines, nonanediamines, decanediamines, undecanediamines, dodecanediamines, tridecanediamines, tetradecanediamines, pentadecanediamines, hexadecanediamines, heptadecanediamines, octadecanediamines, nonadecanediamines, eicosanediamines, heneicosanediamines, docosanediamines, tricosanediamines, tetracosanediamines, pentacosanediamines, hexacosanediamines, heptacosanediamines, octacosanediamines, nonacosanediamines, and triacontyldiamines, including linear and branched isomers of each. In some embodiments, the alkanediamine compounds are α,ω-n-alkanediamines.

Examples of C₁₋₃₀alkyl groups include methyl groups, ethyl groups, propyl groups, butyl groups, pentyl groups, hexyl groups, heptyl groups, octyl groups, nonyl groups, decyl groups, undecyl groups, dodecyl groups, tridecyl groups, tetradecyl groups, pentadecyl groups, hexadecyl groups, heptadecyl groups, octadecyl groups, nonadecyl groups, eicosyl groups, heneicosyl groups, docosyl groups, tricosyl groups, tetracosyl groups, pentacosyl groups, hexacosyl groups, heptacosyl groups, octacosyl groups, nonacosyl groups and triacontyl groups.

Quaternary-amine-containing primary amine compounds for use in the present disclosure include quaternary-amine-containing compounds that comprise one or more triC₁₋₃₀alkylamine groups linked to a C₂₋₃₀alkylamine group, and include (triC₁₋₃₀alkyl)aminoC₂₋₃₀alkylamine compounds, where the three C₁₋₃₀alkyl groups of the (triC₁₋₃₀alkyl)amino group of the quaternary-amine-containing compound can be the same or different.

Examples of C₁₋₃₀alkyl groups are set forth above. Examples of C₂₋₃₀alkylamine groups include ethyleneamine groups, propyleneamine groups, butyleneamine groups, pentyleneamine groups, hexyleneamine groups, heptyleneamine groups, octyleneamine groups, nonyleneamine groups, decyleneamine groups, undecyleneamine groups, dodecyleneamine groups, tridecyleneamine groups, tetradecyleneamine groups, pentadecyleneamine groups, hexadecyleneamine groups, heptadecyleneamine groups, octadecyleneamine groups, nonadecyleneamine groups, eicosyleneamine groups, heneicosyleneamine groups, docosyleneamine groups, tricosyleneamine groups, tetracosyleneamine groups, pentacosyleneamine groups, hexacosyleneamine groups, heptacosyleneamine groups, octacosyleneamine groups, nonacosyleneamine groups, and triacontyl eneamine groups, including linear and branched isomers of each.

In some embodiments, quaternary-amine-containing primary amine compounds that comprises a primary amine group and at least one quaternary amine group are prepared by a synthesis procedure in which a protected tertiary-amine-containing compound comprising a protected primary amine group and at least one tertiary amine group is reacted with an haloalkane (also referred to as an alkyl halide). When a haloalkane reacts with a tertiary amine in a reaction known as the Menshutkin reaction, the primary product is a quaternary ammonium salt. Without wishing to be bound by theory, the tertiary amine acts essentially as a nucleophile, attacking the carbon atom of the haloalkane, replacing the halogen atom and forming a positively charged nitrogen with four alkyl groups attached, creating the quaternary ammonium salt.

Tertiary-amine-containing primary amine compounds comprising a primary amine group and at least one tertiary amine group for use in the present disclosure include tertiary-amine-containing primary amine compounds that comprise a C₂₋₃₀alkanediamine compound in which one amine group is a primary amine group and the other amine group is substituted with two C₁₋₃₀alkyl groups, which that can be the same or different. Examples of C₂₋₃₀alkanediamine compounds and C₁₋₃₀alkyl groups are described above.

Tertiary-amine-containing primary amine compounds for use in the present disclosure include tertiary-amine-containing compounds that comprise one or more (diC₁₋₃₀alkyl)amine groups linked to a C₂₋₃₀alkylamine group, and include (diC₁₋₃₀alkyl)aminoC₂₋₃₀alkylamine compounds, where the two C₁₋₃₀alkyl groups of the (diC₁₋₃₀alkyl)amino group of the tertiary-amine-containing compound can be the same or different. Examples of C₁₋₃₀alkyl groups and C₂₋₃₀alkylamine groups are described above.

Particular examples of tertiary-amine-containing primary amine compounds for use in the present disclosure include 2-(dimethylamino)-1-ethylamine,

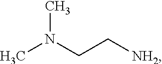

3-(dimethylamino)-1-propylamine,

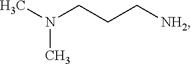

4-(dimethylamino)-1-butylamine,

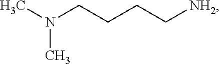

4-(diethylamino)-1-butylamine,

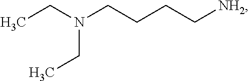

5-(dimethylamino)-1-pentylamine,

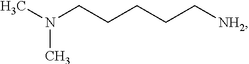

and 5-(dimethylamino)-2-pentylamine,

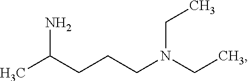

among others.

Haloalkanes for use in the present disclosure include C₁₋₃₀haloalkanes (also referred to as C₁₋₃₀alkyl halides), which may be selected from chloroC₁₋₃₀alkanes (also referred to as C₁₋₃₀alkyl chlorides), bromoC₁₋₃₀alkanes (also referred to as C₁₋₃₀alkyl bromides), and iodoC₁₋₃₀alkanes (also referred to as C₁₋₃₀alkyl iodides). C₁₋₃₀haloalkanes include halomethanes, haloethanes, halopropanes, halobutanes, halopentanes, halohexanes, haloheptanes, halooctanes, halononanes, halodecanes, haloundecanes, halododecanes, halotridecanes, halotetradecanes, halopentadecanes, halohexadecanes, haloheptadecanes, halooctadecanes, halononadecanes, haloeicosanes, haloheneicosanes, halodocosanes, halotricosanes, halotetracosanes, halopentacosanes, halohexacosanes, haloheptacosanes, halooctacosanes, halononacosane and halotriacontanes, including linear and branched isomers of each. Using alternative nomenclature, C₁₋₃₀alkyl halides include methyl halides, ethyl halides, propyl halides, butyl halides, pentyl halides, hexyl halides, heptyl halides, octyl halides, nonyl halides, decyl halides, undecyl halides, dodecyl halides, tridecyl halides, tetradecyl halides, pentadecyl halides, hexadecyl halides, heptadecyl halides, octadecyl halides, nonadecyl halides, eicosyl halides, heneicosyl halides, docosyl halides, tricosyl halides, tetracosyl halides, pentacosyl halides, hexacosyl halides, heptacosyl halides, octacosyl halides, nonacosyl halides and triacontyl halides, including linear and branched isomers of each. It is noted that alkyl iodides (iodoalkanes) produce products having radiopaque iodine anions.

As noted above, in some embodiments, the quaternary-amine-containing compound comprising a primary amine group and at least one quaternary amine group is prepared by a synthesis procedure in which a protected tertiary-amine-containing compound comprising a protected primary amine group and at least one tertiary amine group is reacted with an haloalkane. Examples of protective groups for use in protecting the primary amine group of the tertiary-amine-containing compound include tert-butoxycarbonyl (Boc) groups, 9-fluorenylmethoxycarbonyl (Fmoc) groups, benzyloxycarbonyl (Cbz) groups, acetamide (Ac) groups, triphenylmethylamine (Tr) groups, trifluoroacetyl (TFA) groups, 6-nitroveratryloxycarbonyl (Nvoc) groups, among others.

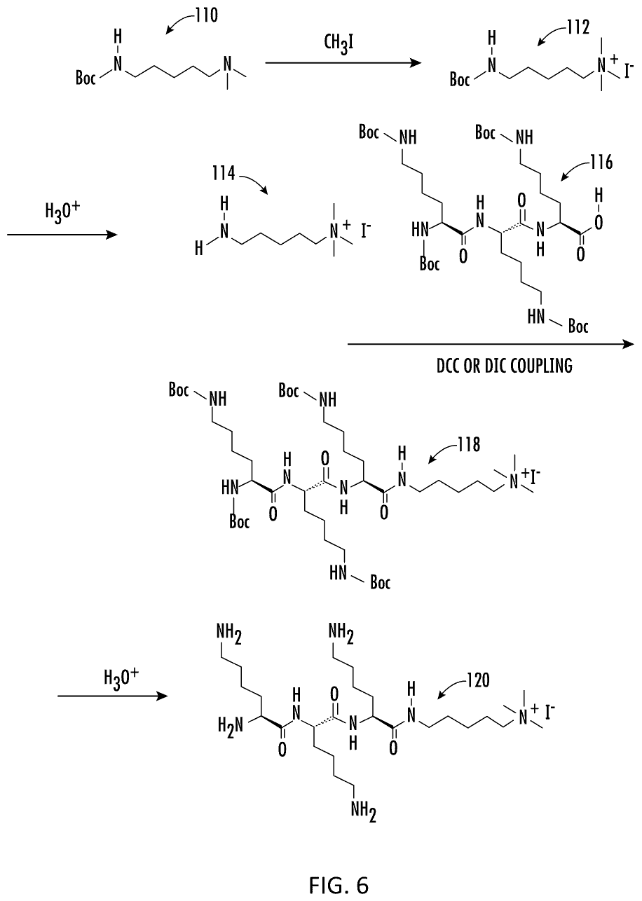

In a particular example shown in FIG. 6, boc-protected 5-dimethylaminopentylamine (110) (also known as tert-butyl N-[5-(dimethylamino)pentyl]carbamate) (CAS #1698868-24-4) is used as starting material to react with iodomethane (CH₃I) (CAS #74-88-4) to obtain a boc-protected quaternary-amine-containing compound, specifically, boc-protected 5-trimethylaminopentylamine (112), with iodine as a counterion. This compound is then deprotected under acidic conditions (designated by H₃O⁺) to attain a free amine version of the quaternary-amine-containing compound, specifically, 5-(trimethylamino)-1-pentylamine (114).

Once obtained, in some embodiments, the quaternary-amine-containing primary amine compound comprising a primary amine group and at least one quaternary amine group is reacted with an amine-protected carboxylic-acid-containing polyamino compound comprising a carboxylic-acid group and a plurality of protected amine groups. In this reaction, the amine group of the quaternary-amine-containing primary amine compound is reacted in an amide coupling reaction with the carboxylic acid group of the protected carboxylic-acid-containing polyamino compound. The amide coupling is typically performed in the presence of a coupling agent. Coupling agents include carbodiimide coupling agents such as N,N′-dicyclohexylcarbodiimide (DCC), 1-ethyl-3-(3-dimethyl propyl) carbodiimide (EDC), 1,3-diisopropylcarbodiimide (DIC), N-hydroxybenzotriazole (HOBt), 2-(1H-Benzotriazole-1-yl)-1,1,3,3-tetramethylaminium tetrafluoroborate (TBTU), oxalyl chloride, pivaloyl chloride, BOP reagent, and/or another coupling agent.

In the particular example shown in FIG. 6, a quaternary-amine-containing primary amine compound, specifically, a (triC₁₋₃₀alkyl)aminoC₂₋₃₀alkylamine compound, more specifically, 5-(trimethylamino)-1-pentylamine (114), is reacted with an amine-protected carboxylic-acid-containing polyamino compound, specifically, an amine-protected poly(amino acid) that comprises a plurality of amino acid residues having primary-amine-containing side groups, more specifically boc-protected trilysine (116) in the presence of a coupling agent, specifically, DCC or DIC. The product of the reaction is an amine-protected quaternary-amine-containing polyamino compound that contains a residue of a quaternary-amine-containing primary amine compound that is linked by an amide group to an amine-protected residue of a carboxylic-acid-containing polyamino compound, specifically, an amine-protected quaternary-amine-containing polyamino compound that contains a residue of a (triC₁₋₃₀alkyl)aminoC₂₋₃₀alkylamine compound that is linked by an amide group to an amine-protected residue of a poly(amino acid) having a plurality of primary-amine-containing side groups, more specifically, an amine-protected quaternary-amine-containing polyamino compound (118) that contains a residue of 5-(trimethylamino)-1-pentylamine that is linked by an amide group to a boc-protected trilysine residue.

After deprotecting the amine groups under acidic conditions, a quaternary-amine-containing polyamino compound is formed that contains a residue of a quaternary-amine-containing primary amine compound that is linked by an amide group to a residue of a carboxylic-acid-containing polyamino compound, specifically, a quaternary-amine-containing polyamino compound that contains a residue of a (triC₁₋₃₀alkyl)aminoC₂₋₃₀alkylamine compound that is linked by an amide group to a residue of a poly(amino acid) having a plurality of primary-amine-containing side groups, more specifically, a quaternary-amine-containing polyamino compound (120) that contains a residue of 5-(trimethylamino)-1-pentylamine that is linked by an amide group to a trilysine residue.

In some embodiments, quaternary-amine-containing primary amine compounds for use in the present disclosure include zwitterionic quaternary-amine-containing primary amine compounds that, in addition to comprising a primary amine group and at least one quaternary amine group, further comprise at least one sulfonate group. Such zwitterionic quaternary-amine-containing primary amine compounds include compounds that comprise a C₂₋₃₀alkanediamine compound in which one amine group is a primary amine and the other amine group is substituted with one sulfoC₁₋₃₀alkyl group and two C₁₋₃₀alkyl groups, which can be the same or different.

Examples of C₂₋₃₀alkanediamine compounds and C₁₋₃₀alkyl groups are provided above. As noted above, some embodiments, the alkanediamine compounds are α,ω-n-alkanediamine compounds.

Examples of sulfoC₁₋₃₀alkyl groups include sulfomethyl groups, sulfoethyl groups, sulfopropyl groups, sulfobutyl groups, sulfopentyl groups, sulfohexyl groups, sulfoheptyl groups, sulfooctyl groups, sulfononyl groups, sulfodecyl groups, sulfoundecyl groups, sulfododecyl groups, sulfotridecyl groups, sulfotetradecyl groups, sulfopentadecyl groups, sulfohexadecyl groups, sulfoheptadecyl groups, sulfooctadecyl groups, sulfononadecyl groups, sulfoeicosyl groups, sulfoheneicosyl groups, sulfodocosyl groups, sulfotricosyl groups, sulfotetracosyl groups, sulfopentacosyl groups, sulfohexacosyl groups, sulfoheptacosyl groups, sulfooctacosyl groups, sulfononacosyl groups and sulfotriacontyl groups.

Quaternary-amine-containing primary amine compounds for use in the present disclosure include zwitterionic quaternary-amine-containing primary amine compounds that comprise one or more (diC₁₋₃₀alkyl)(sulfoC₁₋₃₀alkyl)amino groups linked to a C₂₋₃₀alkyleneamine group, and include zwitterionic (diC₁₋₃₀alkyl)(sulfoC₁₋₃₀alkyl)aminoC₂₋₃₀alkylamine compounds, where the two C₁₋₃₀alkyl groups can be the same or different. Examples of C₁₋₃₀alkyl groups, sulfoC₁₋₃₀alkyl groups and C₂₋₃₀alkylamine groups are set forth above.

In some embodiments, zwitterionic quaternary-amine-containing compounds comprising a primary amine group, at least one quaternary amine group, and at least one sulfonate group may be prepared by a synthesis procedure in which a protected tertiary-amine-containing compound comprising a protected primary amine group and at least one tertiary amine group is reacted with in a ring-opening reaction with a cyclic ester of a hydroxyl sulfonic acid.

Examples of cyclic esters of a hydroxyl sulfonic acid include 1,3-propane sultone (CAS #1120-71-4), which is a cyclic ester of 3-hydroxypropane-1-sulfonic acid, 1,4-butane sultone, which is a cyclic ester of 4-hydroxybutane-1-sulfonic acid, 1,2-ethane sultone, which is a cyclic ester of 2-hydroxyethane-1-sulfonic acid, and prop-1-ene-1,3-sultone, which is a cyclic ester of 3-hydroxyprop-1-ene-1-sulfonic acid, among others. Examples of tertiary-amine-containing primary amine compounds are provided above. Examples of protective groups for use in protecting the primary amine group of the tertiary-amine-containing primary amine compounds are provided above.

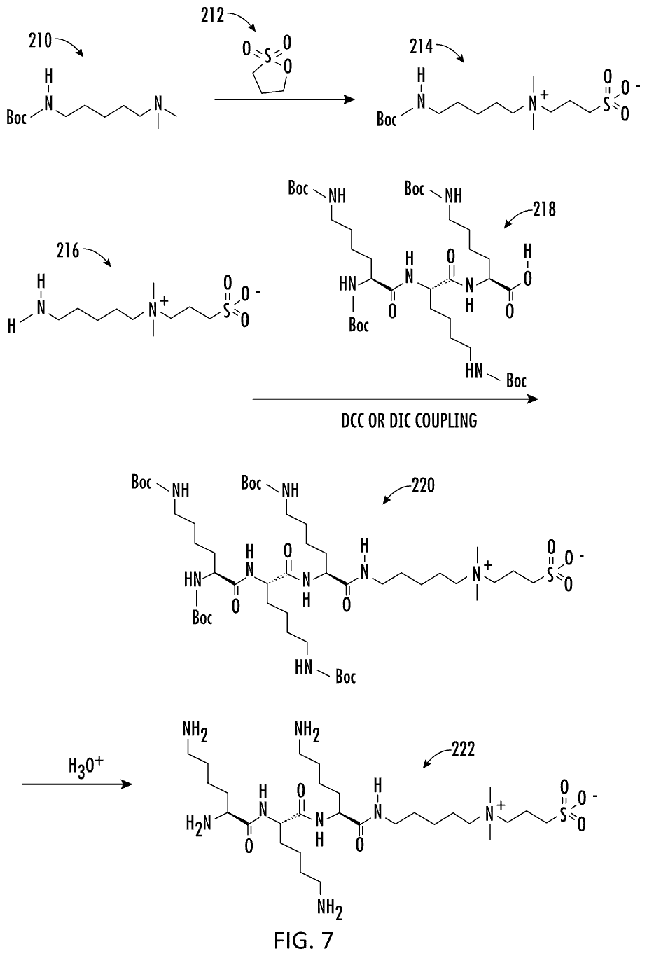

In a particular example shown in FIG. 7, boc-protected 5-dimethylaminopentylamine (210) is used as starting material and undergoes ring-opening reaction with 1,3-propane sultone (212) (CAS #1120-71-4) to obtain a boc-protected zwitterionic quaternary-amine-containing compound, specifically, boc-protected 5-((dimethyl) (3-sulfopropyl)amino)-1-pentylamine (214). This compound is then deprotected under acidic conditions (designated by H₃O⁺) to attain a free amine version of the quaternary-amine-containing compound, specifically, 5-((dimethyl) (3-sulfopropyl)amino)-1-pentylamine (216).

Once obtained, in some embodiments, the zwitterionic quaternary-amine-containing primary amine compound comprising a primary amine group, at least one quaternary amine group and at least one sulfonate group is reacted with an amine-protected carboxylic-acid-containing polyamino compound comprising a carboxylic-acid group and a plurality of protected amine groups. In this reaction, the amine group of the zwitterionic quaternary-amine-containing primary amine compound is reacted in an amide coupling reaction with the carboxylic acid group of the protected carboxylic-acid-containing polyamino compound. The amide coupling is typically performed in the presence of a coupling agent. Examples of coupling agents are provided above.

In the particular example shown in FIG. 7, a zwitterionic quaternary-amine-containing primary amine compound, specifically, a (diC₁₋₃₀alkyl)(sulfoC₁₋₃₀alkyl)aminoC₂₋₃₀alkylamine compound, more specifically, 5-((dimethyl)(3-sulfopropyl)amino)-1-pentylamine (216), is reacted with an amine-protected carboxylic-acid-containing polyamino compound, specifically, an amine-protected poly(amino acid) that comprises a plurality of amino acid residues having primary-amine-containing side groups, more specifically, boc-protected trilysine (218) in the presence of a coupling agent, specifically, DCC or DIC. The product of the reaction is an amine-protected zwitterionic quaternary-amine-containing polyamino compound that contains a residue of a zwitterionic quaternary-amine-containing primary amine compound that is linked by an amide group to an amine-protected residue of a carboxylic-acid-containing polyamino compound, specifically, an amine-protected zwitterionic quaternary-amine-containing polyamino compound that contains a residue of a (diC₁₋₃₀alkyl)(sulfoC₁₋₃₀alkyl)aminoC₂₋₃₀alkylamine compound that is linked by an amide group to an amine-protected residue of a poly(amino acid) having a plurality of primary-amine-containing side groups, more specifically, an amine-protected zwitterionic quaternary-amine-containing polyamino compound (220) that contains a residue of 5-((dimethyl)(3-sulfopropyl)amino)-1-pentylamine that is linked by an amide group to a boc-protected trilysine residue.

After deprotection the amine groups under acidic conditions, a zwitterionic quaternary-amine-containing polyamino compound is formed that contains a residue of a zwitterionic quaternary-amine-containing primary amine compound that is linked by an amide group to a residue of a carboxylic-acid-containing polyamino compound, specifically, a zwitterionic quaternary-amine-containing polyamino compound that contains a residue of a (diC₁₋₃₀alkyl)(sulfoC₁₋₃₀alkyl)aminoC₂₋₃₀alkylamine compound that is linked by an amide group to a residue of a poly(amino acid) having a plurality of primary-amine-containing side groups, more specifically, a zwitterionic quaternary-amine-containing polyamino compound (222) that contains a residue of 5-((dimethyl)(3-sulfopropyl)amino)-1-pentylamine that is linked by an amide group to a trilysine residue.

In other aspects of the present disclosure, products are provided that are the crosslinked reaction product of (a) a quaternary-amine-containing polyamino compound such as those described above and (b) a reactive polymer having reactive groups that are reactive with the amino groups of the quaternary-amine-containing polyamino compound, for example, a reactive multi-arm polymer that comprises a plurality of polymer arms that have reactive end groups that are reactive with the amino groups of the quaternary-amine-containing polyamino compound, including those described above.

In some embodiments, the crosslinked reaction products are hydrogels. As used herein, a “hydrogel,” which may also be referred to herein as a “crosslinked hydrogel,” is a crosslinked polymer that contains water or can absorb water but does not dissolve when placed in water.

In various embodiments, such crosslinked products are visible under X-ray. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on a bench-top micro CT system such as Xtreme CT from Scanco Medical (Wangen-Brüttisellen, Switzerland) or similar.

Such crosslinked products may be formed in vivo (e.g., using a delivery device like that described in FIG. 4), or such crosslinked products may be formed ex vivo and subsequently administered to a subject. Such crosslinked products can be used in a wide variety of biomedical applications, including medical devices, implants, and pharmaceutical compositions.

In various embodiments, the reactive end groups of the reactive multi-arm polymer and the amino groups of the quaternary-amine-containing polyamino compound react with one another via an amide coupling reaction to form a crosslinked product.

Reactive multi-arm polymers in accordance with the present disclosure include reactive multi-arm polymers that comprise a plurality of polymer arms linked to a core region, where the polymer arms comprise a hydrophilic polymer segment. One end of the hydrophilic polymer segment is covalently attached to the core region through a suitable linkage, and a first reactive moiety is covalently attached to an opposite end of the hydrophilic polymer segment through a suitable linkage.

Various reactive multi-arm polymers suitable for linking with the quaternary-amine-containing polyamino compounds described herein are described above, and may have a variety of cores, a variety of hydrophilic polymer segments, and a variety of reactive moieties (including moieties comprising electrophilic groups) as detailed above.

In some aspects of the present disclosure, systems are provided that are configured to deliver (a) a quaternary-amine-containing polyamino compound as described herein and (b) a reactive multi-arm polymer as described herein. The quaternary-amine-containing polyamino compound and the reactive multi-arm polymer are combined under conditions such that the amino groups of the quaternary-amine-containing polyamino compound and the reactive end groups of the reactive multi-arm polymer crosslink with one another. In certain embodiments, those conditions comprise an environment having a basic pH, for example, a pH ranging from about 8.5 to about 12. Such systems can be used to form crosslinked hydrogels, either in vivo or ex vivo.

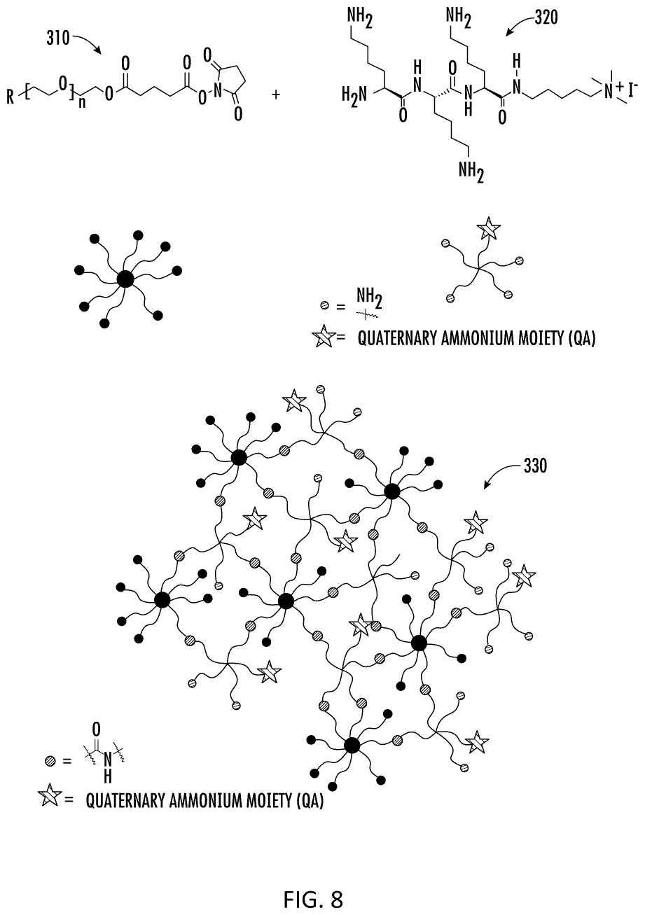

A particular example of a crosslinking reaction is shown in FIG. 8 which employs a succinimidyl-glutaramide-terminated 8-arm PEG (310), where R designates a core region, and may correspond, for example, to a polyol residue such as a tripentaerythritol residue, a hexaglycerol residue, or a sugar residue among others, and n represents and integer which may have a value ranging from 5 to 500. (Note that only a single arm is shown attached to the core in FIG. 8, and the remaining arms are not illustrated.) The succinimidyl-glutaramide-terminated 8-arm PEG (310) is crosslinked with a quaternary-amine-containing polyamino compound in accordance with the present disclosure, specifically, a quaternary-amine-containing polyamino compound (320) that contains a 5-(trimethylamino)-1-pentylamine residue that is linked to a trilysine residue by an amide group. The crosslinking reaction shown is inhibited at acidic pH, but occurs spontaneously at basic pH. The result of the reaction is a crosslinked hydrogel (330), in which residues of the succinimidyl-glutaramide-terminated 8-arm PEG are linked to residues of the quaternary-amine-containing polyamino compound through amide bonds that are formed by reaction between amino groups of the quaternary-amine-containing polyamino compound (320) and succinimidyl ester groups of the succinimidyl-glutaramide-terminated 8-arm PEG (310). In some embodiments, crosslinking is triggered by a change in pH from an acidic pH to a basic pH.

In some aspects of the present disclosure, a system is provided that comprises (a) a first composition that comprises a quaternary-amine-containing polyamino compound as described herein and (b) a second composition that comprises a reactive multi-arm polymer as described herein.

The first composition may be a first fluid composition comprising the quaternary-amine-containing polyamino compound or a first dry composition that comprises the quaternary-amine-containing polyamino compound, to which a suitable fluid such as water for injection, saline, etc. can be added to form a first fluid composition. In addition to the quaternary-amine-containing polyamino compound, the first composition may further comprise additional agents, including therapeutic agents, imaging agents, colorants, tonicity adjusting agents, suspension agents, wetting agents, and pH adjusting agents as described above.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents, including therapeutic agents, imaging agents, colorants, tonicity adjusting agents, suspension agents, wetting agents, and pH adjusting agents as described above.

In some embodiments, the quaternary-amine-containing polyamino compound is initially combined with the reactive multi-arm polymer under conditions where crosslinking between the electrophilic moieties of the reactive multi-arm polymer and the amino groups of the quaternary-amine-containing polyamino compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between same, thereby forming the crosslinked product.

In particular embodiments, the system comprises (a) a first composition that comprises a quaternary-amine-containing polyamino compound as described above, (b) a second composition that comprises a reactive multi-arm polymer as described above, and (c) a third composition, specifically, an accelerant composition, that contains an accelerant that is configured to accelerate a crosslinking reaction between the quaternary-amine-containing polyamino compound and the reactive multi-arm polymer.

The first composition may be a first fluid composition comprising the quaternary-amine-containing polyamino compound that is buffered to an acidic pH or a first dry composition that comprises the quaternary-amine-containing polyamino compound and acidic buffering composition, to which a suitable fluid such as water for injection, saline, etc. can be added to form a first fluid composition comprising the quaternary-amine-containing polyamino compound that is buffered to an acidic pH. In some embodiments, for example, the acidic buffering composition may comprise monobasic sodium phosphate, among other possibilities. The first fluid composition comprising the quaternary-amine-containing polyamino compound may have a pH ranging, for example, from about 3 to about 6.5. In addition to the quaternary-amine-containing polyamino compound, the first composition may further comprise additional agents, including therapeutic agents, imaging agents, colorants, tonicity adjusting agents, suspension agents, wetting agents, and pH adjusting agents as described above.

The second composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the quaternary-amine-containing polyamino compound that is buffered to an acidic pH. In addition to the reactive multi-arm polymer, the second composition may further comprise additional agents, including therapeutic agents, imaging agents, colorants, tonicity adjusting agents, suspension agents, wetting agents, and pH adjusting agents as described above.

In a particular embodiment, the first composition is a first fluid composition comprising the quaternary-amine-containing polyamino compound that is buffered to an acidic pH and the second composition comprises a dry composition that comprises the reactive multi-arm polymer. The first composition may then be mixed with the second composition to provide a prepared fluid composition that is buffered to an acidic pH and comprises the quaternary-amine-containing polyamino compound and the reactive multi-arm polymer. In a particular example, a syringe may be provided that contains the first fluid composition comprising the quaternary-amine-containing polyamino compound that is buffered to an acidic pH, and a vial may be provided that comprises the dry composition (e.g., a powder) that comprises the reactive multi-arm polymer. The syringe may then be used to inject the first fluid composition into the vial containing the reactive multi-arm polymer to form a prepared fluid composition that contains the quaternary-amine-containing polyamino compound and the reactive multi-arm polymer, which can be withdrawn back into the syringe for administration.

The third composition may be a fluid accelerant composition that is buffered to a basic pH or a dry composition that comprises a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 11.5. In addition to the above, the fluid accelerant composition may further comprise additional agents, including those described above.

A prepared fluid composition that is buffered to an acidic pH and comprises the quaternary-amine-containing polyamino compound and the reactive multi-arm polymer as described above (which may include additional agents in some cases), and a fluid accelerant composition that is buffered to basic pH as described above (which may include additional agents in some cases), may be combined form crosslinked hydrogels, either in vivo or ex vivo.

In some particular embodiments of the present disclosure, a kit is provided that include a first reservoir (e.g., a vial or syringe barrel) containing a first composition comprising a quaternary-amine-containing polyamino compound as described herein and an acidic buffer, a second reservoir (e.g., a vial or syringe barrel) containing a second composition comprising containing a reactive multi-arm polymer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the quaternary-amine-containing polyamino compound and the reactive multi-arm polymer, and additional apparatus for combining and delivering the prepared fluid composition and buffered accelerant to a patient.

In more particular embodiments, the kit may comprise a vial containing a reactive multi-arm polymer as described herein in dry (e.g., powdered) form, a first syringe containing a fluid composition comprising a quaternary-amine-containing polyamino compound as described herein that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

The various compositions described in the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO₂, gamma, x-ray or electron beam irradiation.

A prepared fluid composition that is buffered to an acidic pH and comprises the quaternary-amine-containing polyamino compound and the reactive multi-arm polymer as described above and a fluid accelerant composition that is buffered to basic pH as described above, may be combined form crosslinked hydrogels, either in vivo or ex vivo.

In various embodiments, a system is provided that includes one or more delivery devices for delivering the above-described compositions to a subject. In some embodiments, the system may include a delivery device that comprises a first reservoir that contains a first composition that comprises a quaternary-amine-containing polyamino compound as described above and a second reservoir that contains a second composition that comprises a reactive multi-arm polymer that comprises a plurality of electrophilic moieties that are reactive with the amino moieties of the quaternary-amine-containing polyamino compound as described above.

In some embodiments, the system may include a delivery device that comprises a first reservoir that contains a first composition that comprises the quaternary-amine-containing polyamino compound and the reactive multi-arm polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the quaternary-amine-containing polyamino compound and the reactive multi-arm polymer and crosslink with one another to form a crosslinked hydrogel. In particular embodiments, first and second fluid compositions are dispensed simultaneously as described above with reference to FIG. 4.

As previously indicated, first and second compositions as describe herein can be used to form crosslinked hydrogels ex vivo. Crosslinked hydrogels may be provided in any desired form, as described in detail above, and may contain additional agents, including therapeutic agents, imaging agents, colorants, tonicity adjusting agents, suspension agents, wetting agents, and pH adjusting agents as described above.

In some embodiments, kits are provided that include one or more delivery devices for delivering the crosslinked hydrogel to a subject as described above. A particular device for delivering crosslinked hydrogel compositions is described above with reference to FIG. 5.

The first and second compositions, fluid admixtures of the first and second fluid compositions, and ev vivo crosslinked products of the first and second compositions may be injected, implanted, or otherwise introduced in conjunction with a wide variety of medical procedures, including those as described above.

1. A kit for forming an antimicrobial cross-linked hydrogel, the kit comprising:
a multi-arm polymer comprising a core, a plurality of polymer segments having a first end that is covalently attached to the core and a second end comprising a moiety that comprises a reactive group; and
an antimicrobial multifunctional compound that comprises functional groups that are reactive with the reactive group of the multi-arm polymer.

2. The kit of claim 1, wherein the antimicrobial multifunctional compound is a polyamine.

3. The kit of claim 2, wherein the polyamine further comprises a cationic polymer.

4. The kit of claim 1, wherein the antimicrobial multifunctional compound comprises ε-poly-l-lysine n-acyl, ε-poly-l-lysine, ε-poly-l-lysine hydrochloride, ε-poly-l-lysine hydrobromide, ε-poly-l-lysine hydrofluoride, ε-poly-l-lysine hydroiodide, quaternized ε-poly-l-lysine, or any combination thereof.

5. The kit of claim 1, wherein the kit has an absence of another multifunctional compound that comprises functional groups that are reactive with the reactive group of the multi-arm polymer.

6. The kit of claim 5, wherein the another multifunctional compound comprises TLA.

7. The kit of claim 1, wherein the reactive moieties of the multi-arm polymer include moieties that comprise electrophilic groups.

8. An antimicrobial cross-linked reaction product of: (a) a multi-arm polymer comprising a core, a plurality of polymer segments having a first end that is covalently attached to the core and a second end comprising a moiety that comprises a reactive group; and (b) an antimicrobial multifunctional compound that comprises functional groups that are reactive with the reactive group of the multi-arm polymer, wherein the antimicrobial multifunctional compound comprises ε-poly-l-lysine n-acyl, ε-poly-l-lysine, ε-poly-l-lysine hydrochloride, ε-poly-l-lysine hydrobromide, ε-poly-l-lysine hydrofluoride, ε-poly-l-lysine hydroiodide, quaternized ε-poly-l-lysine, or any combination thereof, and wherein a molar ratio of the antimicrobial compound to the multi-arm polymer is in a range from about 0.2:1 to about 10:1.

9. The antimicrobial cross-linked reaction product of claim 8, wherein the cross-linked reaction product has an absence of another multifunctional compound.

10. The antimicrobial cross-linked reaction product of claim 8, wherein the antimicrobial multifunctional compound further comprises ε-poly-L-lysine.

11. A system for forming a hydrogel that comprises (a) a quaternary-amine-containing polyamino compound that comprises a quaternary-amine-containing moiety that comprises a quaternary amine group and a polyamino moiety that comprise a plurality of primary amine groups; and (b) a reactive polymer comprising a plurality of reactive moieties, wherein the reactive moieties are reactive with the plurality of primary amine groups of the quaternary-amine-containing polyamino compound, and wherein the quaternary-amine-containing moiety is coupled to the polyamino moiety through a linkage that comprises an amide group.

12. The system of claim 11, wherein the quaternary-amine-containing moiety comprises a (triC₁-C₃₀alkyl)aminoC₂-C₃₀alkylaminocarbonyl group.

13. The system of claim 11, wherein the quaternary-amine-containing polyamino compound is formed by a method that comprises (a) reacting a quaternary-amine-containing primary amine compound in an amide coupling reaction with an amine-protected carboxylic-acid-containing polyamino compound that comprises a carboxylic-acid group and a plurality of protected amine groups and (b) deprotecting the product of step (a) to form the quaternary-amine-containing polyamino compound.

14. The system of claim 13, wherein the quaternary-amine-containing primary amine compound is a C₂-C₃₀alkanediamine compound that comprises first and second amine groups, wherein the first amine group is a primary amine group and the second amine group is a quaternary amine group that contains three C₁-C₃₀alkyl groups, which can be the same or different.

15. The system of claim 11, wherein the quaternary-amine-containing moiety is a zwitterionic quaternary-amine-containing moiety that comprises the quaternary amine group and a sulfonate group.

16. The system of claim 15, wherein the zwitterionic quaternary-amine-containing moiety comprises a (diC₁-C₃₀alkyl)(sulfoC₁-C₃₀alkyl)aminoC₂-C₃₀alkylaminocarbonyl group.

17. The system of claim 15, wherein the quaternary-amine-containing polyamino compound is formed by a method that comprises (a) reacting a zwitterionic quaternary-amine-containing primary amine compound in an amide coupling reaction with an amine-protected carboxylic-acid-containing polyamino compound that comprises a carboxylic-acid group and a plurality of protected amine groups and (b) deprotecting the product of step (a) to form the quaternary-amine-containing polyamino compound.

18. The system of claim 17, wherein the zwitterionic quaternary-amine-containing primary amine compound is a zwitterionic C₂-C₃₀alkanediamine compound that comprises first and second amine groups in which the first amine group is a primary amine group and second amine group is a quaternary amine group that contains two C₁-C₃₀alkyl groups, which can be the same or different, and one sulfoC₁-C₃₀alkyl group.

19. The system of claim 11, wherein the quaternary amine group comprises an iodide counter-anion.

20. The system of claim 11, wherein the plurality of primary amine groups comprises —(CH₂)ₓ—NH₂ groups where x is 1, 2 3, 4, 5 or 6.
